(19) United States
(12) Patent Application Publication
(10) Pub. No.: US 2022/0369625 A1
(43) Pub. Date: Nov. 24, 2022

(54) ACTIVELY COOLED ORGAN TRANSPLANT SYSTEM

(71) Applicant: ETXMed, Inc., Kaysville, UT (US)

(72) Inventors: Kent F. Beck, Layton, UT (US); James Malmstrom, Kaysville, UT (US); Patrick D. McNally, Salt Lake City, UT (US)

(21) Appl. No.: 17/748,014

(22) Filed: May 18, 2022

Related U.S. Application Data
(60) Provisional application No. 63/190,684, filed on May 19, 2021.

(51) Int. Cl. A01N 1/02

(52) U.S. Cl. CPC A01N 1/0247; A01N 1/0252

(57) ABSTRACT

An organ transplant system provides a pumping and refrigeration unit and a sterile disposable set to maintain a desired temperature of an organ during transplantation and to position the organ adjacent a surgical site during an organ transplantation procedure. Handling and exposure of the organ is minimized while maintaining position and condition of the organ.

### THE FIELD OF THE INVENTION

The present invention relates to organ transplant. In particular, examples of the present invention relate to an actively cooled system for transplanting organs which maintains proper temperature regulation of organs during transport and transplantation, and which also reduces handling time and transplant time for the organ.

### BACKGROUND

There continues to be a high demand for organ transplants to improve quality and duration of life for persons with damaged organs. While the number of organ transplant procedures is primarily limited by the number of suitable donor organs, the suitability of the donor organs and the outcomes of the transplant procedures may also be limited by the transport and handling of the organ between harvest and transplant.

### BRIEF DESCRIPTION OF THE DRAWINGS

Non-limiting and non-exhaustive examples of the present invention are described with reference to the following figures, wherein like reference numerals refer to like parts throughout the various views unless otherwise specified.

FIG. 1 is a drawing of an organ transplant system.

FIG. 2 is a schematic drawing which shows a top view of the pumping unit.

FIG. 3 is a drawing of the cooled transplant pouch.

FIG. 4 is a drawing of the organ transplant system.

FIG. 5 is a drawing showing the use of the cooled transplant pouch.

FIG. 6 is a drawing showing the use of the cooled transplant pouch.

FIG. 7 is a drawing showing the use of the cooled transplant pouch.

FIG. 8 is a drawing showing the use of the cooled transplant pouch.

FIG. 9 is a drawing showing the use of the cooled transplant pouch.

FIG. 10 is a drawing of another cooled transplant pouch showing the cooled transplant pouch in a partially assembled state.

FIG. 11 is another drawing of the cooled transplant pouch of FIG. 10 in a partially assembled state.

FIG. 12 is a drawing of the cooled transplant pouch of FIG. 10.

FIG. 13 is a drawing of the cooled transplant pouch of FIG. 10.

FIG. 14 is a drawing of an organ transplant system.

FIG. 15 is a drawing of an organ transplant system.

FIG. 16 is a drawing of an organ transplant system.

Corresponding reference characters indicate corresponding components throughout the several views of the drawings. Unless otherwise noted, the drawings have been drawn to scale. Skilled artisans will appreciate that elements in the figures are illustrated for simplicity and clarity. For example, the dimensions of some of the elements in the figures may be exaggerated relative to other elements to help improve understanding of various examples of the present invention. Also, common but well-understood elements that are useful or necessary in a commercially feasible embodiment are often not depicted in order to facilitate a less obstructed view of these various embodiments of the present invention.

It will be appreciated that the drawings are illustrative and not limiting of the scope of the invention which is defined by the appended claims. The examples shown each accomplish various different advantages. It is appreciated that it is not possible to clearly show each element or advantage in a single figure, and as such, multiple figures are presented to separately illustrate the various details of the examples in greater clarity. Similarly, not every example need accomplish all advantages of the present disclosure.

### DETAILED DESCRIPTION

In the following description, numerous specific details are set forth in order to provide a thorough understanding of the present invention. It will be apparent, however, to one having ordinary skill in the art that the specific detail need not be employed to practice the present invention. In other instances, well-known materials or methods have not been described in detail in order to avoid obscuring the present invention.

In the above disclosure, reference has been made to the accompanying drawings, which form a part hereof, and in which are shown by way of illustration specific implementations in which the disclosure may be practiced. It is understood that other implementations may be utilized, and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, such feature, structure, or characteristic may be used in connection with other embodiments whether or not explicitly described. The particular features, structures or characteristics may be combined in any suitable combination and/or sub-combinations in one or more embodiments or examples. It is appreciated that the figures provided herewith are for explanation purposes to persons ordinarily skilled in the art.

As used herein, “adjacent” refers to near or close sufficient to achieve a desired effect. Although direct contact is common, adjacent can broadly allow for spaced apart features.

As used herein, the singular forms “a,” and, “the” include plural referents unless the context clearly dictates otherwise.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be such as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a number or numerical range endpoint by providing that a given value may be “a little above” or “a little below” the number or endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Dimensions, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

The disclosure particularly describes a system for improving the handling of donor organs between harvesting and transplant. The present system may preserve the organ in optimal physical and thermal conditions through the transplant surgery. The present system may also protect the organ and minimize handling and movement of the organ during the transplant procedure.

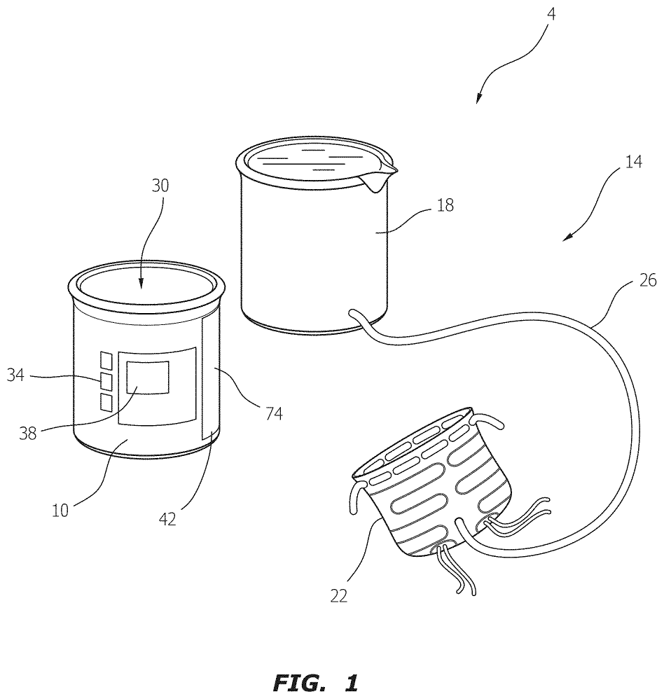

Turning now to FIG. 1, an example organ transplant system, generally indicated at 4, may include a pumping unit 10 and a disposable transplant set 14 used with the pumping unit 10 during transplant of an organ. The disposable transplant set 14 may include a reservoir 18, a cooled transplant pouch, such as a pouch 22, and a set of cooling tubes 26 connecting the reservoir 18 to the transplant pouch 22. The pumping unit 10 may include a cavity 30 which is sized and shaped to receive the reservoir 18. The pumping unit 10 may include a refrigeration unit which is disposed adjacent the cavity 30 and which may be operable to cool fluid in the reservoir 18 when the reservoir 18 is disposed in the cavity 30. The refrigeration unit may be a thermoelectric (Peltier) cooling unit or a liquid/gas phase cooling unit. The pumping unit 10 may include a pump which receives at least one of the tubes 26 to circulate cooled fluid between the reservoir 18 and the cooled transplant pouch 22. In one example, the pump may be a peristaltic pump or a linear or rotary pump which acts upon the exterior of the tubing 26 to pump fluid through the tubing 26. Such a pump allows the tubing 26 to be loaded into the pump without requiring a break in the tubing 26. The pumping unit 10 may include a door or insertion slot/channel which allows the tubing 26 to be loaded into position at the pump.

The pumping unit 10 may include a control panel 34 and display/user interface 38 which allow a user to select a desired operational setting for the pumping unit 10. The control panel 34 and user interface 38 may also have a pre-set or automatic operational setting which is configured to maintain an organ in optimal temperature conditions. The pumping unit 10 may include a computer controller which interfaces with the refrigeration unit, the pump, one or more temperature sensors, the control panel 34, and the display/user interface 38 and which receives user inputs and controls the operation of the pumping unit 10. A user may select a desired temperature for the organ within the cooled transplant pouch 22, for example. The pumping unit display 38 may show the selected temperature. The computer controller may sense the temperature of the returning fluid from the cooled transplant pouch 22 via a temperature sensor which is disposed in contact with the tubing carrying fluid returning from the cooled transplant pouch 22, calculate the temperature within the cooled transplant pouch 22 from the sensed temperature, and display the cooled transplant pouch temperature on the display 38. Alternatively, the computer controller may have a pre-set temperature for the organ and may automatically keep the organ at the pre-set temperature in response to a user turning the system on. The display/user interface 38 may also be simplified and provide one or more indicators such as LED indicators to indicate that the system is on, and that the organ is within a target temperature range.

The computer controller may also operate the refrigeration unit and the pump. In the example system, the computer controller may be connected to a temperature sensor adjacent the reservoir cavity 30 and to the refrigeration unit. The computer controller may operate the refrigeration unit at a duty cycle which keeps cooling fluid inside of the reservoir 18 at a set point which may be at or near the cooling fluid freezing point. The cooling fluid may be circulated through the tubing 26 by the pump to cool the organ within the cooled transplant pouch 22. In the example system, the reservoir 18 may be connected to the cooled transplant pouch 22 with a supply tube and a return tube, indicated as tubing 26. The pump may pump fluid from the reservoir 18 through the supply tube, through cooling fluid channels formed in the cooled transplant pouch 22, through the return tube, and back into the reservoir 18. The computer controller may be connected to a temperature sensor which is in contact with the return tubing 26 to thereby sense the temperature of the cooling fluid returning from the cooled transplant pouch 22. From the sensed temperature of the return cooling fluid, the computer controller may sense the temperature of the organ being transplanted and can adjust the flow rate of the pump to achieve the desired organ temperature within the cooled transplant pouch 22. If the temperature of the returning cooling fluid is too low, the computer controller can decrease the flow rate of the pump. If the temperature of the returning cooling fluid is too high, the computer controller may increase the flow rate of the pump. In such a system, the flow rate of the pump is adjusted to maintain the organ at the desired temperature and the duty cycle/operational load of the refrigeration unit is adjusted to accommodate the cooling load of the organ and keep the reservoir 18 at a desired temperature. The reservoir 18 need not be kept at its freezing point, and may be operated at a desired temperature below the target organ temperature so as to provide a temperature differential between the cooling fluid and the target organ temperature to allow the pumped fluid to cool the organ to the desired temperature.

In the example disposable transplant set 14, the reservoir 18, cooled transplant pouch 22, and cooling tubes 26 may be formed into a single piece. Having these components formed as a single integral disposable set 14 avoids leaks and keeps the cooled transplant pouch 22 and associated components sterile. As described, the disposable transplant set 14 including the reservoir 18, the cooled transplant pouch 22, and the tubing 26 may be provided within a sterile package as an integral unit. The disposable transplant set 14 may be placed within the pumping unit 10 without disconnecting or connecting fluid tubing 26 and without compromising the sterility of the cooled transplant pouch 22, the tubing 26, or the tubing pathways within the cooled transplant pouch 22. Possible contamination within the cavity 30 or within the pump is not transferred to the cooling circuit or to the cooled transplant pouch 22. The temperature sensors, pump, refrigeration unit, and control components are all part of the pumping unit 10 and not the disposable set 14; minimizing the cost of the disposable set 14.

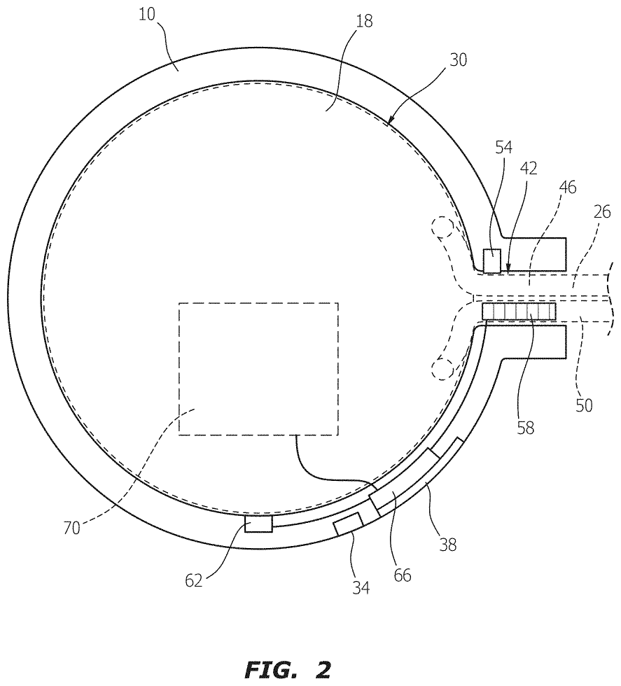

FIG. 2 shows a top schematic view of the pumping unit 10. The pumping unit cavity 30 can be seen with the reservoir 18 shown in dashed lines inside of the reservoir 30. The reservoir 18 may be designed to be a slip fit into the cavity 30 so that it can be easily loaded while maintaining good heat transfer between the reservoir 18 and cavity 30. The cooling tubes 26 may extend outwardly from the reservoir 18 and may be located in an insertion channel 42 which allows the cooling tubes 26 to pass outside of the pumping unit 10 without requiring a break in the cooling tubes 26. The cooling tubes 26 may include a return tube 46 and a supply tube 50. The pumping unit 10 may include a return fluid temperature sensor 54 which is positioned adjacent the installed return tube 46. The pumping unit 10 may include a pump 58 with a pump head disposed adjacent the supply tube 50 so that the pump 10 can work on the supply tube 50 to pump cooled fluid through the supply tube 54, the cooled transplant pouch 22, and the return tube 46. The pumping unit 10 may also include a supply temperature sensor 62 which may be disposed to measure temperature from the reservoir 18 as shown or from the supply tube 50. The pumping unit may also include a refrigeration unit 70. Each of these electrical components may be connected electrically to the computer controller 66 and the control panel 34 and display/user interface 38 are used to control operation of the pumping unit. The computer controller 66 may control operation of the refrigeration unit 70 based on temperature readings from the supply temperature sensor 62. The computer controller 66 controls operation of the pump 58 based on temperature readings from the supply temperature sensor 62 and/or the return temperature sensor 54 to regulate the temperature of an organ in the cooled transplant pouch 22.

FIG. 1 shows how the example pumping unit 10 may include a tube mounting clamp 74 which is used to hold the supply tube 50 in position against the pump 58 and to hold the return tube 46 in place at the return temperature sensor 54. Where the pump 58 is a peristaltic pump or other pump which acts upon the outside of the tubing 26, the tube mounting clamp 74 may securely hold the tube 27 against the pump head to allow the pump 58 to operate. The tube mounting clamp 74 may be a door which closes over the cooling tube insertion channel 42 or may be a block or clamp which engages the pumping unit 10 adjacent the cooling tube insertion channel 42.

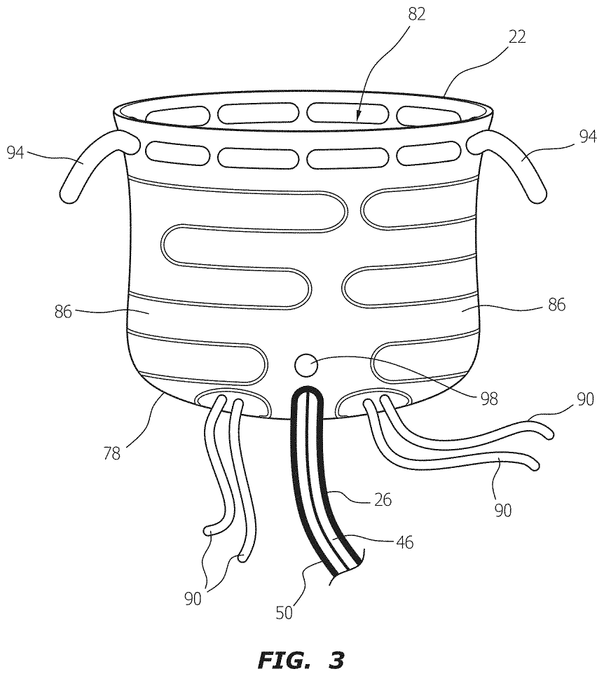

FIG. 3 shows a drawing of the cooled transplant pouch 22. The cooled transplant pouch 22 may have a closed bottom end 78 and an open top end 82. The cooling fluid supply tube 50 and return tube 46 may enter the cooled transplant pouch 22 near the closed bottom end 78. Cooling fluid passages 86 may be formed in the walls of the cooled transplant pouch 22. In one example, the cooled transplant pouch 22 may be formed with an inner wall and an outer wall with cooling passages 86 formed between the inner wall and outer wall. The cooling fluid passages 86 wind back and forth around the cooled transplant pouch 22 to cover a portion of the cooled transplant pouch. In the example, the cooling fluid passages 86 may cover a majority of the surface of the cooled transplant pouch 22. The cooling fluid passages 86 may form multiple cooling circuits which each begin at the supply tube 50, cover a section of the cooled transplant pouch 22, and end at the return tube 46 so that fluid from the reservoir 18 is pumped through these fluid passages to cool the transplant pouch 22 and thereby cool an organ held in the cooled transplant pouch 22.

The cooled transplant pouch 22 may include fasteners such as suspension ties 90 which are attached to the bottom end 78 of the transplant pouch 22. During use, the suspension ties 90 may be attached to a support frame at the operating table or even to the surgical drape on the patient. The suspension ties 90 may be used to position the cooled transplant pouch 22 while the organ is grafted into the patient's body. The cooled transplant pouch 22 may also include a closure 94 such as a drawstring or loop clamp which is disposed adjacent the top opening 82. The closure 94 may be used to substantially close the top opening 82 and retain an organ within the cooled transplant pouch 22 during a procedure. The cooled transplant pouch 22 may also include a temperature indicator 98 which provides a quick visual indicator to the medical staff regarding the temperature of an organ held within the cooled transplant pouch 22. The temperature indicator may be providing an actual temperature of an organ disposed with the pouch 22, or may simply indicate whether the organ temperature has exceeded a predetermined threshold. In the example cooled transplant pouch 22, the temperature indicator may change color or otherwise change appearance if the organ temperature has exceeded a temperature limit and thereby indicate a time requirement for completion of the transplant surgery.

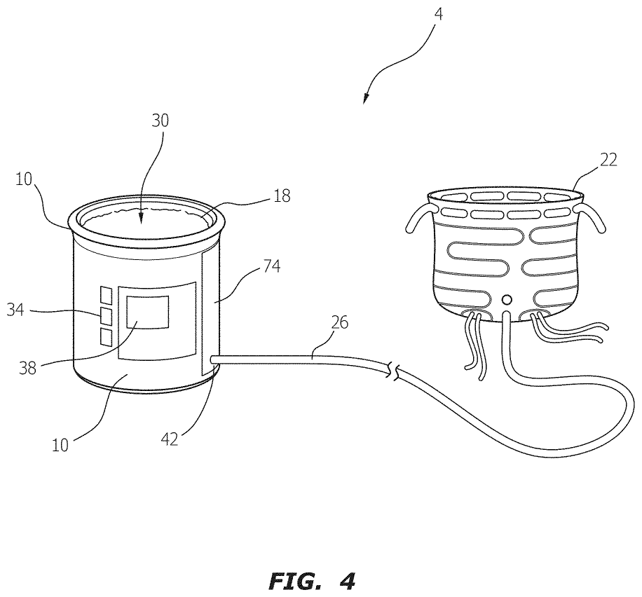

FIG. 4 shows a drawing of the organ transplant system 4 ready for use. The pumping unit 10 is cleaned and readied for surgery. The disposable set 14 may be removed from sterile packaging and the reservoir 18 is placed into the cavity 30 in the pumping unit 10. The fluid tubes 26 may be loaded into the pumping unit 10 and a tube mounting clamp 74 is closed over the tubes 26 to hold the tubes 26 in place relative to the pump 58 and temperature sensors 54, 62. Sterile saline solution is added to the reservoir 18 and the pumping unit 10 is operated to cool the saline solution for use as the cooling fluid to cool the organ. The organ is placed into the cooled transplant pouch 22 and the closure 94 is closed around the organ as desired for the transplant surgery. The system is advantageous because the organ is only exposed to the disposable transplant set 14 which is sterile. Additionally, sterile liquid is introduced as cooling fluid.

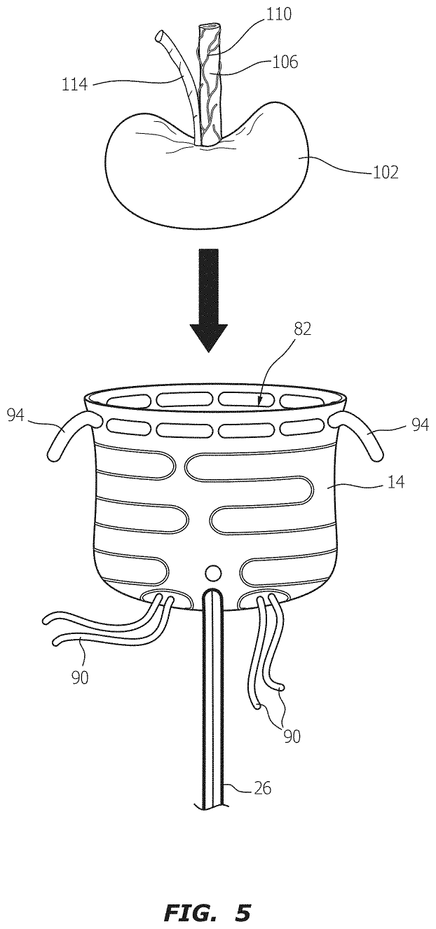

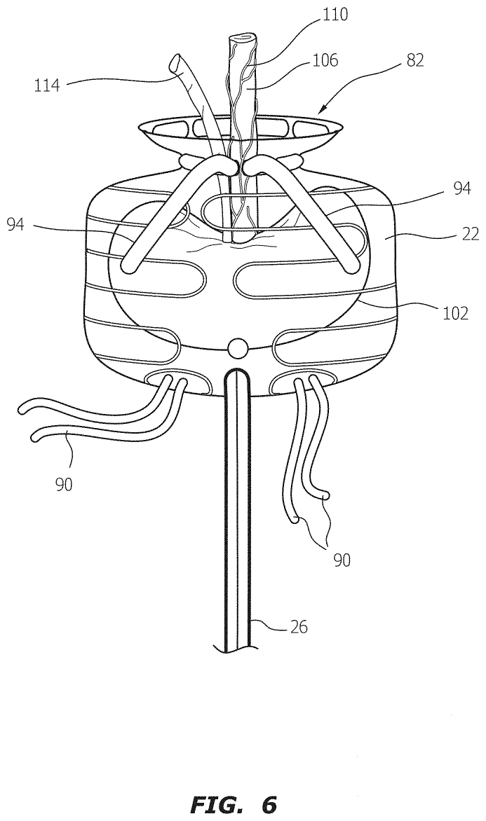

FIGS. 5 through 9 show the use of the cooled transplant pouch 22 during a kidney transplant surgery as one representative example. It will be appreciated that the steps for transplanting other organs may vary. FIG. 5 shows an organ 102, a kidney in this example, which has been prepared for transplant surgery. The kidney 102 includes an artery 106, a vein 110, and a ureter 114 which must be grafted to the recipient's body during transplant. The kidney 102 is aligned so that the artery 106, vein 110, and ureter 114 are oriented away from the cooled transplant pouch opening 82 and the kidney 102 is then placed into the cooled transplant pouch 22 through the opening 82 so that the artery 106, vein 110, and ureter 114 extend out of the opening 82. The orientation of the organ 102 inside of the cooled transplant pouch 22 is generally selected to maximize access to parts of the organ 102 such as arteries 106 and veins 110 which need to be grafted to the recipient. Once the organ 102 is placed into the cooled transplant pouch 22 in a desired orientation, the closure fastener 94 is used to close the opening 82 around the parts of the organ 102 which need to be grafted. The opening 82 need not be closed tightly. Leaving some space around the parts of the organ which need to be grafted reduces damage to the organ and allows these parts (e.g., artery 106, vein 110, and ureter 114) to be moved while still supporting the organ 102 within the cooled transplant pouch 22. The closed cooled transplant pouch 22 is shown in FIG. 6.

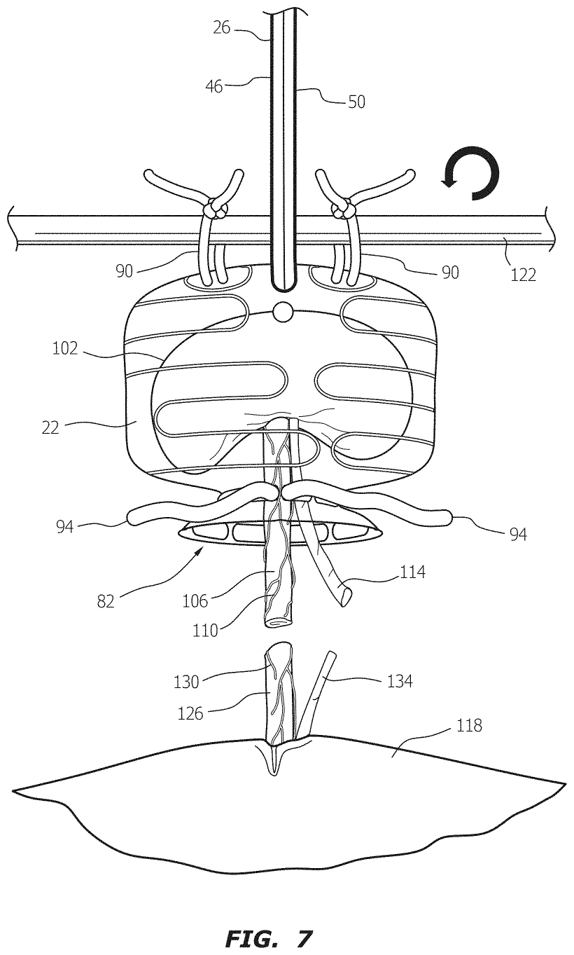

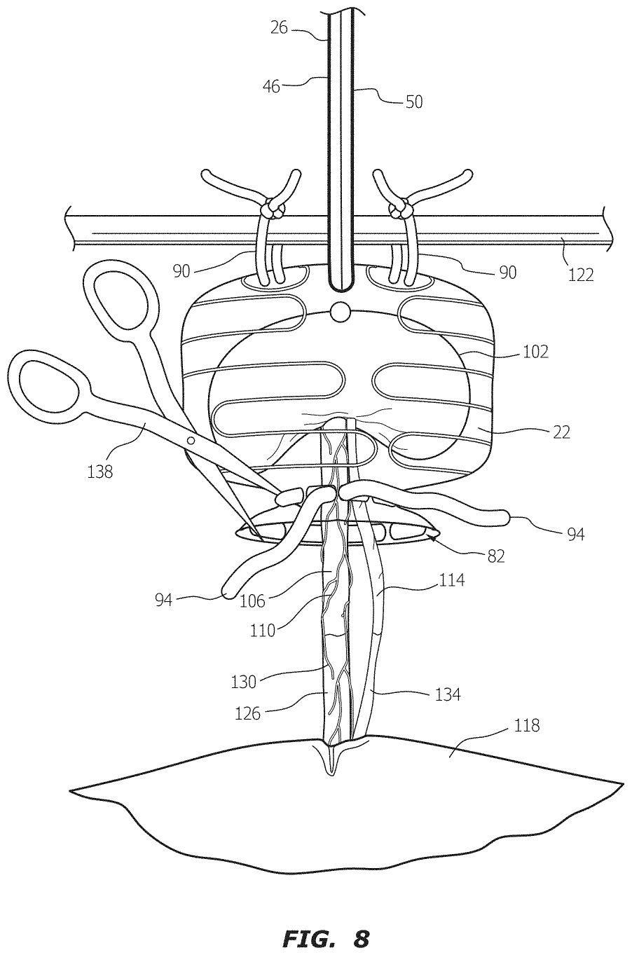

The cooled transplant pouch 22 is then rotated and moved into position above a patient 118 who is the recipient of the organ 102. Typically, the cooled transplant pouch 22 is inverted so that the opening 82 is disposed downwardly towards the patient 118. The suspension ties 90 may be used to attach the cooled transplant pouch 22 to a surgical support frame 122, the surgical drape or some other structure (such as the surgical port) to position the cooled transplant pouch 22 during the transplant surgery as shown in FIG. 7. The surgical support frame 122 is placed so as to position the cooled transplant pouch 22 so that the organ artery 106, vein 110, and ureter 114 are positioned adjacent the corresponding patient artery 126, vein 130, and ureter 134. The surgeon may then attach the organ artery 106, vein 110, and ureter 114 to the corresponding patient artery 126, vein 130, and ureter 134 as is shown in FIG. 8. In many cases, the ureter 114 may be attached to the patient after removal of the organ from the cooled transplant pouch 22 and verification of functionality of the organ. Throughout the transplant surgery, the pump 58 may circulate cooling fluid at a desired temperature through the cooled transplant pouch 22 via the supply tube 50 and return tube 46, maintaining the organ 102 at the desired temperature.

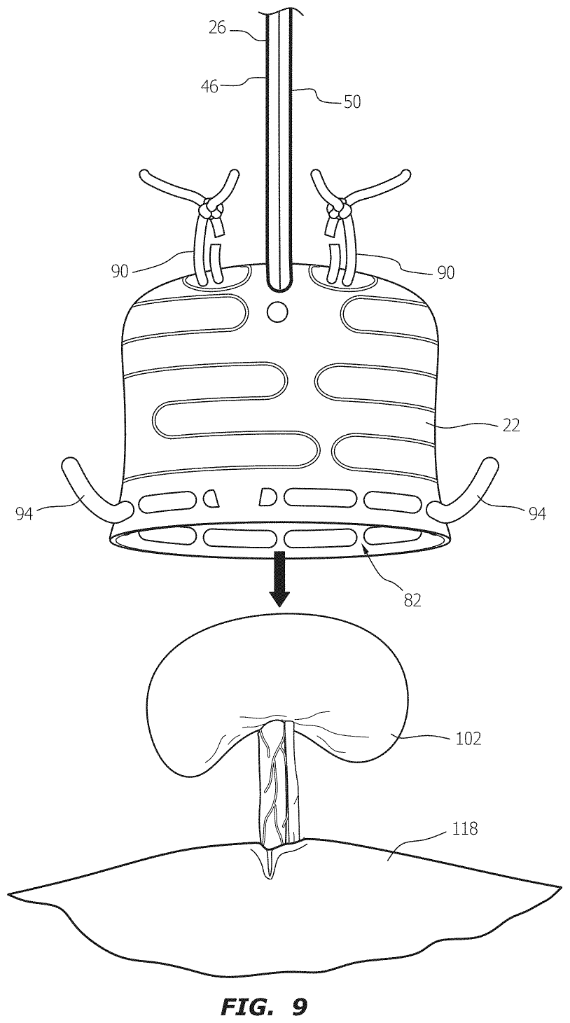

Once the surgeon has completed grafting the organ artery 106, vein 110, and ureter 114 to the corresponding patient artery 126, vein 130, and ureter 134, they may remove the organ 102 from the cooled transplant pouch. As shown in FIG. 8, the surgeon may use scissors 138 to cut the closure fastener 94; allowing the surgeon to expand the opening 82 and release the organ 102 from the cooled transplant pouch 22 as shown in FIG. 9. Alternatively, the surgeon may otherwise release the fastener to allow the opening to open more fully. The surgeon may move the surgical support frame 122 out of the way or otherwise disconnect the suspension ties, position the organ 102 in place in the patient 118, and complete the transplant surgery. The surgeon may cut the suspension ties 90 and remove the cooled transplant pouch 22 from the surgical support frame 122.

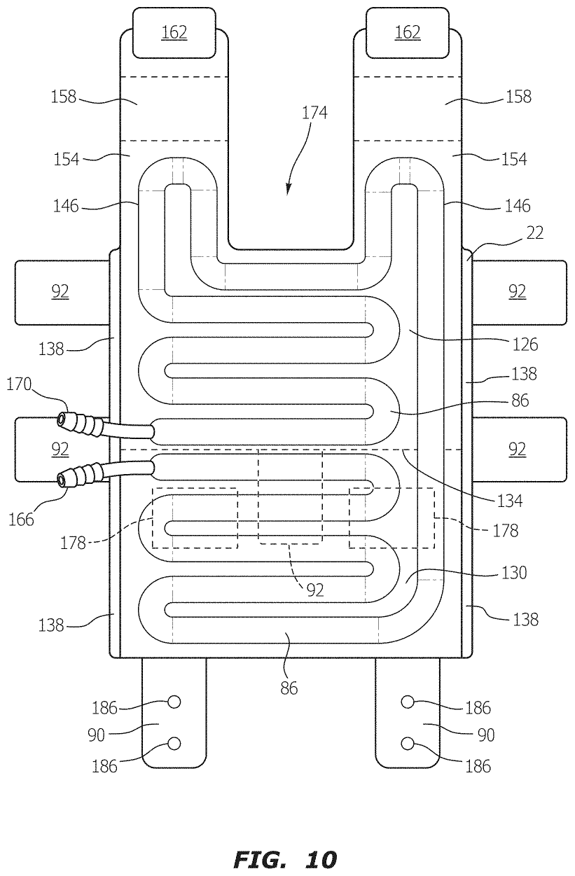

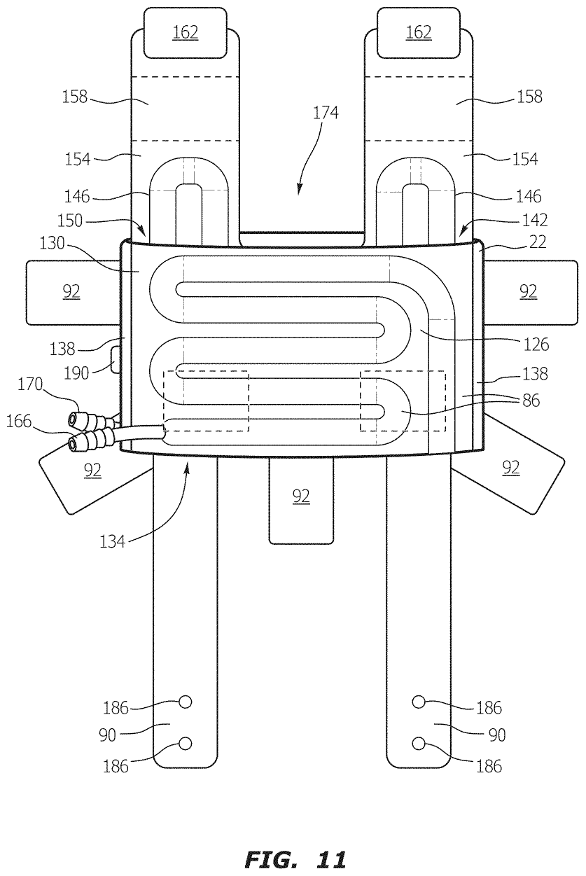

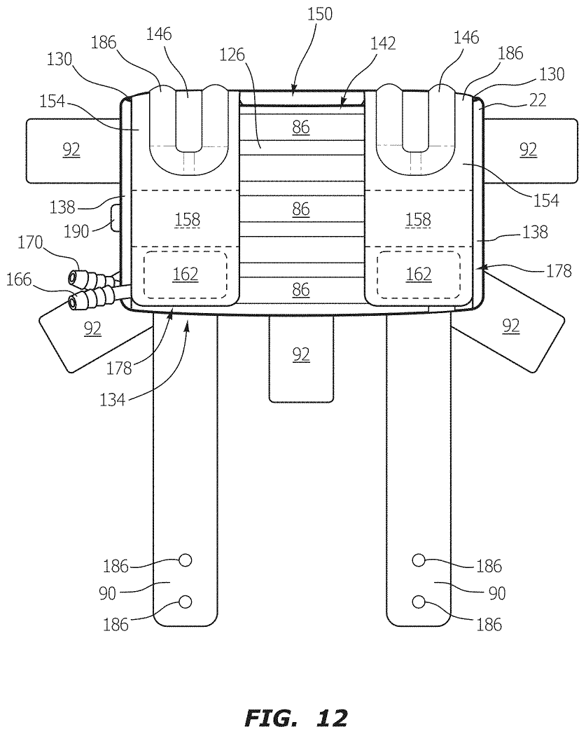

FIGS. 10 through 12 show another cooled transplant pouch 22. FIG. 10 shows the cooled transplant pouch 22 in an open state to illustrate the cooling fluid passages 86 formed within the walls of the cooled transplant pouch. FIG. 11 shows the cooled transplant pouch 22 in a ready to use state. FIG. 12 shows the cooled transplant pouch 22 in a closed configuration, such as during an organ transplant procedure.

The walls of the example cooled transplant pouch 22 may be made from a thermoplastic, such as thin (about 2 mil) low density polyethylene plastic. As shown in FIG. 10, the cooled transplant pouch 22 includes a first pouch side panel 126 and a second pouch side panel 130 which are connected about a center section 134. The first side panel 126 and second side panel 130 are folded towards each other and the adjacent lateral edges 138 may be joined together to form an organ cavity 142. The cooled transplant pouch 22 may include closure straps 146 extending from an edge of the first side panel 126 generally opposite the center section 134. The closure straps 146 are preferably cooled so that they can cool the organ disposed therein. In the assembled cooled transplant pouch 22, the cooled closure straps 146 extend away from the opening 150. The cooled closure straps 146 include a first, cooled section 154 disposed adjacent the first side panel 126, a second, intermediate section 158 attached to the first, cooled section 154, and a fastener 162 attached to the second, intermediate section 158.

The cooled transplant pouch 22 may include a fluid inlet 166 and a fluid outlet 170. The fluid inlet 166 and fluid outlet 170 may be Luer lock fittings or barbed tubing connectors. Alternatively, the fluid inlet 166 and fluid outlet 170 may be ends of fluid inlet and outlet tubes which are joined to the cooled transplant pouch 22. The fluid inlet 166 and fluid outlet 170 may be fluidly connected to a cooling fluid passage 86 in the cooled transplant pouch 22 such that cooling fluid is contained within a fluid passageway formed by an inlet/supply tube 50, the fluid inlet 166, the fluid passage 86, the fluid outlet 170, and an outlet/return tube 46.

The fluid passage 86 may weave back and forth across the first side panel 126, the second side panel 130, the first cooled closure strap 146, and the second cooled closure strap 146. The example fluid passage 86 may be configured such that it bends back and forth across the first side panel 126, forms a loop through the first cooled section 154 of the first cooled closure strap 146, forms a loop through the first cooled section 154 of the second cooled closure strap 146, crosses the center section 134, and bends back and forth across the second side panel 130. This configuration of the cooling passage 86 is advantageous for several reasons. The cooling fluid passage configuration creates a single flow path rather than a branched or parallel flow path which may result in uneven flow. The cooling fluid passage configuration may also minimize the number of times that the cooling fluid passage crosses bends in the cooled transplant pouch 22 and thereby minimizes pressure drop within the cooling fluid passage.

In the example cooled transplant pouch 22, the fluid inlet 166 and the fluid outlet 170 are placed on opposite sides of the center section 134 of the body of the cooling fluid pouch 22. This results in the fluid inlet 166 and fluid outlet 170 being located adjacent each other on the bottom of the cooled transplant pouch 22 opposite the opening 150. This places the fluid inlet and the fluid outlet farthest away from the surgical site and minimizes their crowding of the surgical site.

In the example cooled transplant pouch 22, the first side panel 126, second side panel 130, first cooled closure strap 146, and second cooled closure strap 146 may be formed by sealing two layers of low-density polyethylene thermoplastic together. The two layers of plastic may be attached together around the edges and around the cooling fluid passage 86 to seal them together. The two layers of plastic may also be sealed to the fluid inlet 166 and fluid outlet 170 so that the fluid inlet 166, fluid outlet 170, and cooling fluid passage 86 are sealed and leak free. Manufacturing processes such as heat sealing, ultrasonic welding, or laser welding may be used to fuse the two layers of plastic together and create the fluid passages 86. The two layers of plastic used to form the body of the cooled transplant pouch 22 may be clear so that the cooled transplant pouch 22 is predominantly clear. This allows the surgeon to more easily observe and maintain proper orientation of the organ during the transplant procedure, as well as monitor the color of the organ being transplanted. For most organs, a particular orientation of the organ should be maintained during the transplant procedure so that the various blood vessels, ducts, and tissue are maintained. The color of the organ can be important as an indicator of how the organ is withstanding the procedure.

The cooled transplant pouch 22 may include a space 174 between the cooled closure straps 146 along the edge of the first side panel 126. The body of the cooled transplant pouch 22 may include a first fastener 178 and a second fastener 178. The first and second fasteners 178 may be located on the bottom of the cooled transplant pouch 22 as shown in FIG. 10 and are indicated in dashed lines. The first and second fasteners 178 may be disposed adjacent the center section 134 of the body of the cooled transplant pouch 22. The first and second fasteners 178 engage the fasteners 162 on the cooled closure straps 146 to close the cooled closure straps 146 across the opening 150 and retain an organ in the cooled transplant pouch 22.

FIG. 11 shows a drawing of the cooled organ transplant pouch 22 in an assembled configuration. Relative to FIG. 10, the second side panel 130 has been folded upwardly towards the first side panel 126 and the lateral edges 138 have been attached together to form an organ cavity 146 between the first side panel 126 and the second side panel 130. The lateral edges may be pleated or left unattached to each other in a small area adjacent the center section 134 to allow more space at the bottom of the cooled transplant pouch 22, to allow an organ to enter the organ cavity 142 more easily, and to help prevent kinking the cooling fluid passage 86 where it traverses the center section 134.

For a cooled transplant pouch 22 designed for kidney transplant, the organ cavity 142 formed between the first side panel 126 and the second side panel 130 may be about 15 cm wide, about 10 cm tall, and accommodate about 5 cm of depth between the first side panel 126 and the second side panel 130. The cooled closure straps may be about 5 cm wide and about 10 cm long and the space 174 between the first cooled closure strap 146 and the second closure strap 146 may be about 5 cm wide, or between about 3 cm wide and about 5 cm wide.

The first and second fasteners 178 are disposed on the outside of the front (second side panel 130) of the cooled transplant pouch 22 adjacent the center section 134 which now forms the bottom of the cooled transplant pouch 22. The first and second fasteners 178 interact with the first and second fasteners 162 on the first and second cooled closure straps 146 respectively. A cooled closure strap fastener 162 is attached to a body fastener 178 to thereby secure them together and thereby hold the cooled closure strap 146 closed across the top opening 150 of the cooled transplant pouch 22, as is shown in FIG. 12. Accordingly, the first and second fasteners 162 on the cooled closure straps 146 and the first and second fasteners 178 on the body may be interacting halves of a fastener system that cooperate to close the cooled closure straps 146. The fasteners 162 may be a section of hook fastener material and the fasteners 178 may be a section of loop fastener material (or vice versa). The fasteners 162 may be a magnet and the fasteners 178 may be a magnet or metal component (or vice versa). The fasteners 162 may be a section of tape and the fasteners 178 may be a section of tape receptive material (or vice versa). The fasteners 162 and the fasteners 178 may be snap fasteners.

The second, intermediate section 158 of the cooled closure straps 146 may be a stretchable material. Such can be formed, for example, by forming pleats into the layers of plastic used to make the pouch 22. The intermediate section 158 may be an elastic material which provides a moderately low elastic force and allows the cooled closure straps 146 to stretch a few cm without applying an overly large force. This allows a user to more easily close the cooled closure straps 146 around an organ within the organ cavity 142 and hold the organ snugly within the organ cavity 142 without applying a significant force to the organ. The stretchable cooled closure straps 146 also allow the organ cavity to expand to provide more space to an organ held within the organ cavity. For a kidney transplant, the kidney will expand once blood flow is permitted through the kidney. A stretchable section within the cooled closure straps 146 allows the organ to expand within the organ cavity without becoming overly compressed by the cooled transplant pouch 22 and allows a surgeon to verify proper functioning of the organ without removing it from the cooled transplant pouch 22.

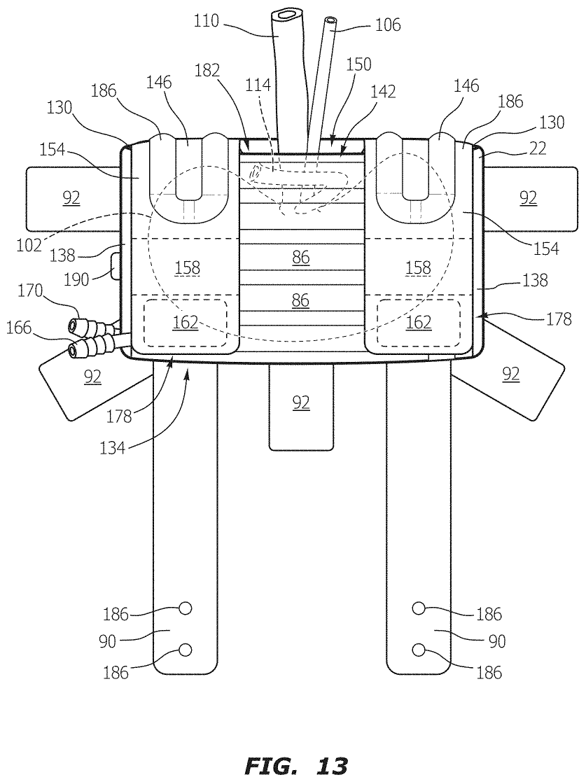

FIG. 13 shows the cooled transplant pouch 22 with a kidney 102 held in the organ cavity 142. The cooled closure straps 146 are extended across the opening 150 to secure the kidney 102 in the organ cavity. The opening 150 is not closed by the cooled closure straps 146. Rather, a space of between about 2 cm and about 4 cm is left between the upper edges of the first side panel 126 and the second side panel 130. The cooled closure straps 146 bridge across the opening 150 but need not be used to pull the edges along the opening 150 together. This allows the cooling fluid passages 86 to remain straighter and minimizes restriction to the fluid flow. This also creates a surgical window 182 framed between the first cooled closure strap 146, the second cooled closure strap 146, the upper edge of the first side panel 126, and the upper edge of the second side panel 130.

The cooled transplant pouch 22 is preferably sized so that the organ 102 fills the organ cavity 142 from top to bottom. The cooled closure straps 146 are thus in contact with the top of the organ 102 when they are in the closed position shown in FIG. 13 and when cooling fluid is flowing through the cooling fluid channels 86. This keeps the top of the organ 102 near the surgical window 182 and optimizes access to the artery 106, vein 110, and duct 114. The dimensions of the cooled transplant pouch 22 may be adjusted according to the size of the intended type of organ 102. The cooled transplant pouch 22 is typically small and compact and is near in dimension to the intended type of organ 102. In the example cooled transplant pouch 22, the wall thickness of the pouch with fluid in the cooling fluid passages 86 is about 6 mm. Cooling fluid in the cooling fluid passages 86 provides cushioning for the organ.

Some parts of the cooled transplant pouch 22, such as an elastic intermediate section 158 of the cooled closure strap 146, fasteners 178, fasteners 162, fluid inlet 166, or fluid outlets 170 may be an opaque material. Otherwise, the cooled transplant pouch 22 may be predominantly clear to allow the surgeon to observe orientation and condition of the organ 102 during use. For a kidney, the ureter 114 may be tucked out of the way into the cooled transplant pouch 22 while the artery 106 and vein 110 are attached. The cooled transplant pouch 22 allows the surgeon to still see the ureter and verify that the kidney is properly oriented during the transplant procedure. After grafting the artery 106 and vein 110, the surgeon may allow blood flow to the kidney 102 and verify that the kidney is working properly by observing urine flow out of the ureter 114. The elastic intermediate section 158 of the cooled closure strap 146 allows the kidney to expand without requiring removal from the cooled transplant pouch 22.

The cooled transplant pouch 22 may include suspension ties 90. In the example transplant pouch 22, the suspension ties 90 may be attached to the bottom of the transplant pouch 22 adjacent the center section 134. The suspension ties 90 are formed with connection points which allow use of the suspension ties to attach the transplant pouch 22 to a surgical frame during use or otherwise support the cooled transplant pouch. The illustrated suspension ties 90 may have fastener elements 186 which may be used to attach the cooled transplant pouch 22 to a surgical frame, support arm, surgical drape or otherwise support the transplant pouch 22 via the suspension ties. The fastener elements 186 may be cooperating halves of a snap fastener, a clamp or loop, or other fastener elements such as described herein, such as hook and loop fastener elements, adhesive fastener elements, or magnetic fastener elements.

The cooled transplant pouch 22 may include grasp tabs 92 in addition to or as an alternative to the suspension ties. The grasp tabs 92 are attached to the cooled transplant pouch 22 around the sides and bottom of the pouch 22 so that they extend from the cooled transplant pouch 22 around its edges when the pouch 22 is in an assembled state as is shown in FIGS. 11 through 13. When an organ 102 is held in the cooled transplant pouch 22 ready for surgery, the grasp tabs 92 extend from the sides and bottom of the cooled transplant pouch 22. The grasp tabs 92 may be held by clamps on a surgical frame or other supporting structure to position the cooled transplant pouch 22 during a transplant surgery. In one example use, the grasp tabs 92 may be held by an arm of a robotic surgery device while the organ 102 is attached to a patient during a surgery. The robotic surgery device may include an arm or support structure with one or more clamps that attach to one or more of the grasp tabs 92 to hold the cooled transplant pouch 22 during surgery. If desired, the robotic surgery device may then suture the organ 102 to the patient during the surgery.

The cooled transplant pouch 22 may also include a temperature indicator 190. The temperature indicator 190 may be an LED indicator which displays a first color such as green if the temperature of the organ 102 is below a predetermined temperature and a second color such as red if the temperature of the organ 102 exceeds a predetermined temperature.

During use, the suspension ties 90 may be attached to a surgical frame, port or surgical drape adjacent the transplant site. This stabilizes the cooled transplant pouch 22 and prevents movement of the organ 102. This allows for easier work and reduced chance of pulling on sutured tissue during further work. The cooled transplant pouch 22 is closely fit to the organ 102 and maximizes the surgeon access to the organ 102 while still covering the organ 102 and separating the organ from the recipient body. The cooling fluid in the cooling passages 86 shield the organ 102 from contact damage and provide temperature regulation and cooling. The fasteners 162 on the cooled closure straps 146 may be attached to the body of the cooled transplant pouch 22 at fastener locations 178 which are near the bottom of the transplant pouch 22 away from the surgical window 182. This minimizes the likelihood that the fasteners 162 interfere with the surgery or are accidentally unfastened. When the grafting of the organ 102 is completed, the fasteners 162 are located on the cooled transplant pouch 22 at a location far away from the surgical window and grafted artery 106, vein 110, and duct 114. The surgeon may release the fasteners 162 and dispense the organ 102 from the cooled transplant pouch 22 from the side of the transplant pouch 22 which faces away from the surgical window and graft site. The surgeon may release the fasteners 162 easily and without risk of disturbing the grafted organ. The cooled closure straps 146 are then easily moved away from the opening 150 and the organ 102 may be dispensed from the cooled transplant pouch 22 for final placement into the recipient body.

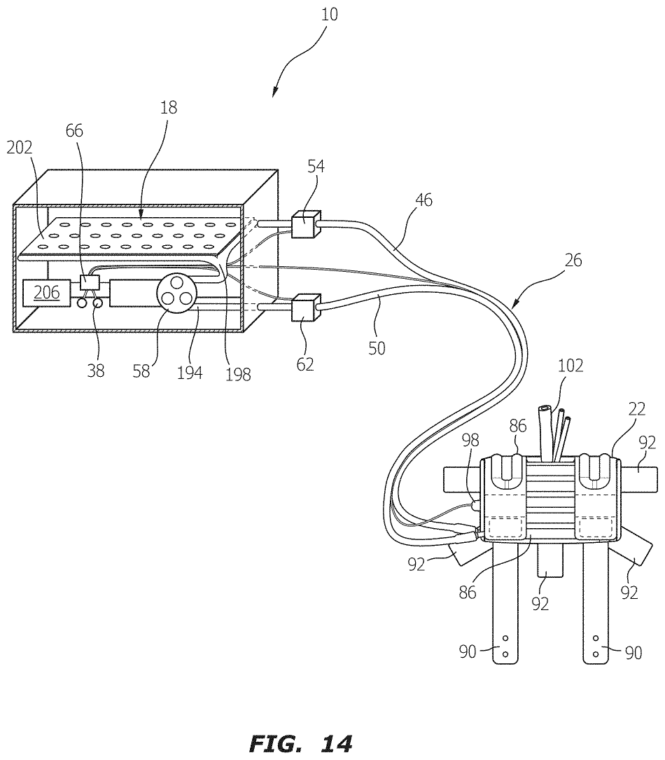

FIG. 14 shows a transplant system 4 which includes a cooled transplant pouch 22. The transplant system includes a pumping unit 10 which is connected to a cooled transplant pouch 22 by cooling tubes 26. The cooled transplant pouch 22 is as described above. The pumping unit 10 includes a peristaltic pump 58 which engages a section of pump tubing 194 to act thereupon and circulate fluid. The peristaltic pump 58 pumps cooling fluid from a reservoir 18 and into a supply tube 50 and delivers cooling fluid to the cooled transplant pouch 22. The fluid returns to the pumping unit 10 from the cooled transplant pouch 22 through a return tube 46. The return tube 46 discharges cooling fluid into a reservoir 18. The peristaltic pump 58 draws cooling fluid from the reservoir 18 via a pump inlet 198. In use, a person would fill the reservoir 18 with a combination of sterile ice and saline solution. The ice may be a slush made from frozen saline or an ice made from frozen water. The heat of freezing of the ice in the slush mixture provides cooling capacity during the transplant surgery. The pumping unit includes a screen or baffle 202 located in the reservoir 18. The screen 202 prevents pieces of ice from entering the pump inlet 198 and only allows liquid to enter the pump inlet 198.

The flow path of liquid through the reservoir 18 may be controlled to ensure that properly cooled liquid is being provided. For example, the return fluid may be returned to the reservoir at several locations, such as in a showerhead configuration so as to avoid a channel of warmed water making its way back to the pump without adequate mixing with the water in the reservoir.

The transplant system 10 may include electronic components used to control the operation of the pumping unit 10 temperature indicator 190. The pump 58 is connected to a computer controller 66 which controls operation of the pump 58. The computer controller 66 is connected to a power source 206 which provides power to operate the electrical components of the transplant system. The power source 206 may be a battery, transformer, or connection to external power such as a building mains power supply. The transplant system 10 may include a supply temperature sensor 62 which is disposed in thermal connection with the cooling fluid in the supply tube 50 and a return temperature sensor 54 which is disposed in thermal connection with the cooling fluid in the return tube 46. The temperature sensors may be electrically connected to the computer controller 66. The computer controller 66 is also connected to the temperature indicator 98. The computer controller 66 is electrically connected to a user interface 38 which may include buttons and a display screen to allow a user to control and observe the operation of the pumping unit 10. The user interface 38 may allow the user to turn the pumping unit 10 on or off, to control temperature set points, to control pumping speed/flow, and to observe the operational characteristics of the pumping unit 10 such as the cooling fluid temperature(s).

In operation, the reservoir 18 may be filled with a sterile mixture of ice and saline. The pump 58 draws chilled liquid from the reservoir 18 and circulates the chilled liquid through the supply tube 50, cooling fluid passages 86, return tube 46, and into the reservoir 18. The returned cooling fluid is typically a few degrees warmer than the supply cooling fluid. The supply cooling fluid will typically be close to the freezing/melting point of the ice and saline mixture. The returned cooling fluid is chilled by melting the ice. The computer controller 66 monitors the temperature of the supply cooling fluid via the supply temperature sensor 62 and monitors the temperature of the return cooling fluid via the return temperature sensor 54. If the temperature of the return cooling fluid is above a predetermined set point, the computer controller 66 increases the operational speed of the pump-to-pump cooling fluid at a higher rate. If the temperature of the return cooling fluid is below a predetermined set point, the computer controller 66 may decrease the operation speed of the pump-to-pump cooling fluid at a lower rate. The computer controller 66 may have a lookup table stored in memory where pump speed is correlated to return cooling fluid temperature. The computer controller 66 may operate the pump 58 at a speed determined from the lookup table and may further increase or decrease the pump speed according to a proportional and/or integral correction factor according to a measured difference in return fluid temperature from a predetermined (desired) return cooling fluid temperature. The temperature indicator 98 is electrically or optically connected to the computer controller. The computer controller 66 may operate the temperature indicator 98 according to the temperature of the return cooling fluid. If the temperature of the return cooling fluid is below a predetermined indicator temperature set point, the computer controller 66 operates the temperature indicator to display a first color of light such as green light. If the temperature of the return cooling fluid is above the indicator temperature set point, the computer controller 66 operates the temperature indicator 98 to display a second color of light such as red or orange light. In one example, the temperature indicator is an RGB LED or a multicolor LED. In another example, the temperature indicator is a small optical display connected to a light source via a fiber optic cable. The light source may be an LED in or near the pumping unit 10 and the optical display may be a simple lens that disperses the light and makes it visible to the surgeon.

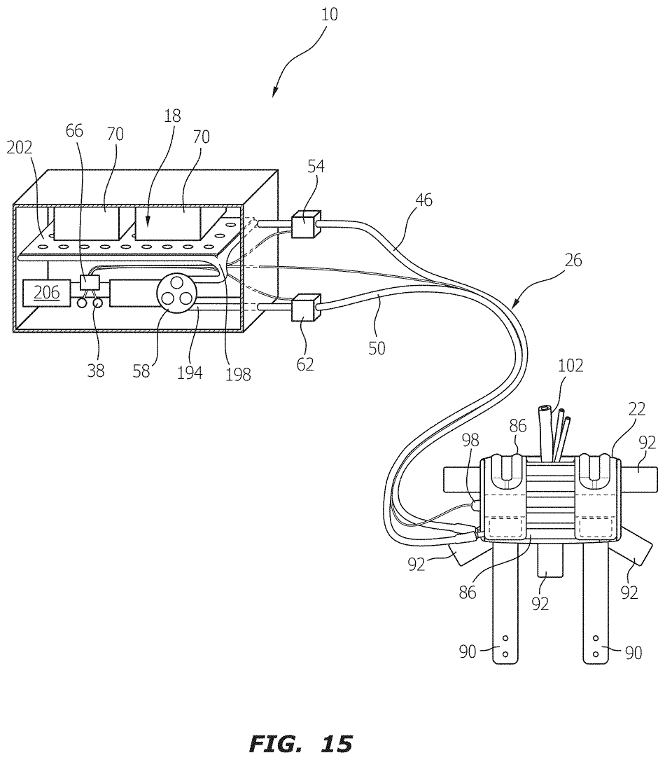

FIG. 15 shows another transplant system which includes a cooled transplant pouch 22. The transplant system includes a pumping unit 10 which is connected to a cooled transplant pouch 22 by cooling tubes 26. The cooled transplant pouch 22 is as described above. The pumping unit 10 includes a peristaltic pump 58 which engages a section of pump tubing 194 to act thereupon and circulate fluid. The peristaltic pump 58 pumps cooling fluid from a reservoir 18 and into a supply tube 50 and delivers cooling fluid to the cooled transplant pouch 22. The fluid returns to the pumping unit 10 from the cooled transplant pouch 22 through a return tube 46. The return tube 46 discharges cooling fluid into a reservoir 18. The peristaltic pump 58 draws cooling fluid from the reservoir 18 via a pump inlet 198. The reservoir is thermally connected to one or more refrigeration units 70 such as thermoelectric (Peltier) cooling units 70 which are operated to cool the cooling fluid in the reservoir 18. The refrigeration units 70 may also include necessary ancillary equipment, such as heat sinks and cooling fans to remove heat. The thermoelectric cooling units are electrically connected to the computer controller 66 and are controlled by the computer controller to achieve a desired cooling fluid temperature. The computer controller 66 may measure the temperature of cooling fluid in the reservoir 18 via the supply temperature sensor 62 (which may be placed in or near the reservoir). The computer controller may turn the refrigeration units 70 on if the temperature of the cooling fluid in the reservoir 18 is above a predetermined set point, and may turn the refrigeration units 70 off if the temperature of the cooling fluid in the reservoir 18 is below a predetermined set point, or may operate the refrigeration units 70 at a load or duty cycle according to the temperature of the cooling fluid in the reservoir 18. In use, a person would fill the reservoir 18 with a sterile fluid such as water or saline solution. The refrigeration unit 70 would cool the cooling fluid in preparation of and during the transplant surgery. In such a configuration, the pumping unit may not need to include a screen or baffle 202 located in the reservoir 18, although such a screen 202 may still be employed to prevent pieces of ice from forming on the refrigeration units 70 and entering the pump inlet 198 and only allows liquid to enter the pump inlet 198. Such a system does away with the need for sterile ice or slush and may be operated with readily available sterile saline solution.

The transplant system includes electronic components used to control the operation of the pumping unit 10 temperature indicator 190. The pump 58 may be connected to a computer controller 66 which controls operation of the pump 58. The computer controller 66 may be connected to a power source 206 which provides power to operate the electrical components of the transplant system. The power source 206 may be a battery, transformer, or connection to external power, such as a building mains power supply. The transplant system may include a supply temperature sensor 62 which is disposed in thermal connection with the cooling fluid in the supply tube 50 and a return temperature sensor 54 which is disposed in thermal connection with the cooling fluid in the return tube 46. The temperature sensors may be electrically connected to the computer controller 66. The computer controller 66 may also be connected to the temperature indicator 98. The computer controller 66 may be electrically connected to a user interface 38 which may include buttons and a display screen to allow a user to control and observe the operation of the pumping unit 10. The user interface 38 may allow the user to turn the pumping unit 10 on or off, to control temperature set points, to control pumping speed/flow, and to observe the operational characteristics of the pumping unit 10, such as the cooling fluid temperature(s).

In operation, the reservoir 18 may be filled with a sterile mixture of ice and saline. The pump 58 draws chilled liquid from the reservoir 18 and circulates the chilled liquid through the supply tube 50, cooling fluid passages 86, return tube 46, and into the reservoir 18. The returned cooling fluid is typically a few degrees warmer than the supply cooling fluid. The supply cooling fluid will typically be close to the freezing/melting point of the ice and saline mixture. The returned cooling fluid is chilled by melting the ice. The computer controller 66 monitors the temperature of the supply cooling fluid via the supply temperature sensor 62 and monitors the temperature of the return cooling fluid via the return temperature sensor 54. If the temperature of the return cooling fluid is above a predetermined set point, the computer controller 66 increases the operational speed of the pump-to-pump cooling fluid at a higher rate. If the temperature of the return cooling fluid is below a predetermined set point, the computer controller 66 decreases the operation speed of the pump-to-pump cooling fluid at a lower rate. The computer controller 66 may have a lookup table stored in memory where pump speed is correlated to return cooling fluid temperature. The computer controller 66 may operate the pump 58 at a speed determined from the lookup table and may further increase or decrease the pump speed according to a proportional and/or integral correction factor according to a measured difference in return fluid temperature from a predetermined (desired) return cooling fluid temperature. The temperature indicator 98 may be electrically or optically connected to the computer controller. The computer controller 66 may operate the temperature indicator 98 according to the temperature of the return cooling fluid. If the temperature of the return cooling fluid is below a predetermined indicator temperature set point, the computer controller 66 may operate the temperature indicator to display a first color of light, such as green light. If the temperature of the return cooling fluid is above the indicator temperature set point, the computer controller 66 operates the temperature indicator 98 to display a second color of light, such as red or orange light. In one example, the temperature indicator is an RGB LED or a multicolor LED. In another example, the temperature indicator is a small optical display connected to a light source via a fiber optic cable. The light source may be an LED in or near the pumping unit 10 and the optical display may be a simple lens that disperses the light and makes it visible to the surgeon.

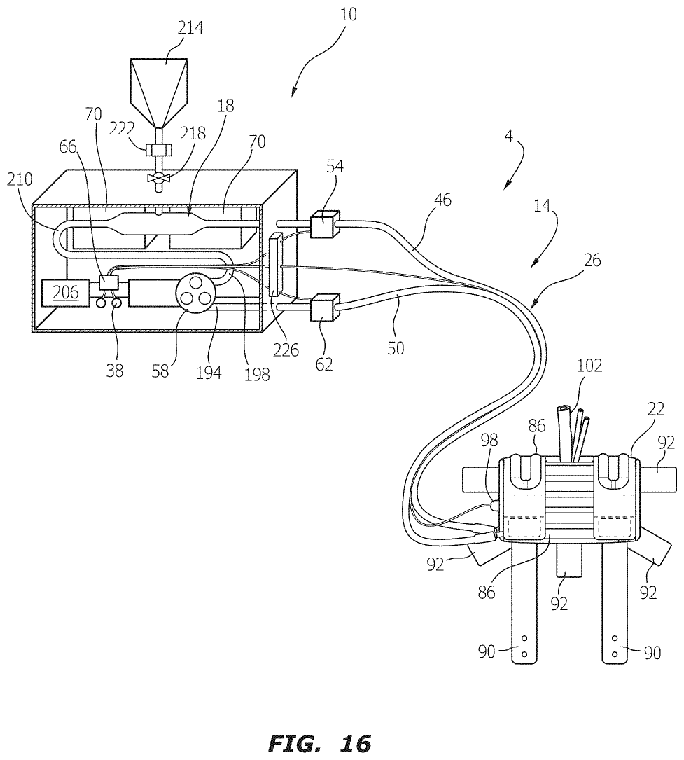

FIG. 16 shows another transplant system 4 which includes a cooled transplant pouch 22. The transplant system includes a pumping unit 10 which is connected to a cooled transplant pouch 22 by cooling tubes 26. The cooled transplant pouch 22 is as described above. The pumping unit 10 may include a peristaltic pump 58 which engages a section of pump tubing 194 to act thereupon and circulate fluid. The peristaltic pump 58 pumps cooling fluid into a supply tube 50 and delivers cooling fluid to the cooled transplant pouch 22. The fluid returns to the pumping unit 10 from the cooled transplant pouch 22 through a return tube 46. The pumping unit is designed to remove or significantly reduce the size of the reservoir 18. The return tube 46 may discharge cooling fluid into a small internal reservoir 18 or a cooling loop 210 which is thermally connected to refrigeration units 70. The reservoir/cooling loop may be an enlarged section of tubing or a cooling block with cooling passages. The peristaltic pump 58 draws cooling fluid from the reservoir 18 and across the refrigeration units 70 via a pump inlet 198. The refrigeration units 70 may be thermoelectric (Peltier) cooling units 70 which are operated to cool the cooling fluid in the reservoir 18/cooling loop 210. The refrigeration units 70 may also include necessary ancillary equipment, such as heat sinks and cooling fans to remove heat. The thermoelectric cooling units are electrically connected to the computer controller 66 and are controlled by the computer controller to achieve a desired cooling fluid temperature. The computer controller 66 may measure the temperature of cooling fluid via the supply temperature sensor 62. The computer controller may turn the refrigeration units 70 on if the temperature of the cooling fluid in the reservoir 18 is above a predetermined set point, and may turn the refrigeration units 70 off if the temperature of the cooling fluid in the reservoir 18 is below a predetermined set point, or may operate the refrigeration units 70 at a load or duty cycle according to the temperature of the cooling fluid in the reservoir 18. In use, a person would fill the cooling fluid circuit including any reservoir 18, cooling tubes 26, and cooled transplant pouch 22 with a sterile fluid, such as water or saline solution. The refrigeration unit 70 would cool the cooling fluid in preparation of and during the transplant surgery. The sterile fluid may be an IV bag 214 of saline which may be connected to the pumping unit 10 via a fill and purge valve 218 which allows a user to fill the cooling circuit with sterile liquid and purge air from the cooling circuit. Such a system does away with the need for sterile ice or slush and may be operated with readily available sterile saline solution.

The transplant system 4 may include electronic components used to control the operation of the pumping unit 10 temperature indicator 190. The pump 58 is connected to a computer controller 66 which controls operation of the pump 58. The computer controller 66 is connected to a power source 206 which provides power to operate the electrical components of the transplant system. The power source 206 may be a battery, transformer, or connection to external power, such as a building mains power supply. The transplant system includes a supply temperature sensor 62 which is disposed in thermal connection with the cooling fluid in the supply tube 50 and a return temperature sensor 54 which is disposed in thermal connection with the cooling fluid in the return tube 46. The temperature sensors are electrically connected to the computer controller 66. The computer controller 66 is also connected to the temperature indicator 98. The computer controller 66 is electrically connected to a user interface 38, which may include buttons and a display screen to allow a user to control and observe the operation of the pumping unit 10. The user interface 38 may allow the user to turn the pumping unit 10 on or off, to control temperature set points, to control pumping speed/flow, and to observe the operational characteristics of the pumping unit 10, such as the cooling fluid temperature(s).

In operation, the reservoir 18 may be filled with a sterile mixture of ice and saline. The pump 58 draws chilled liquid from the reservoir 18 and circulates the chilled liquid through the supply tube 50, cooling fluid passages 86, return tube 46, and into the reservoir 18. The returned cooling fluid is typically a few degrees warmer than the supply cooling fluid. The supply cooling fluid will typically be close to the freezing/melting point of the ice and saline mixture. The returned cooling fluid is chilled by melting the ice. The computer controller 66 monitors the temperature of the supply cooling fluid via the supply temperature sensor 62 and monitors the temperature of the return cooling fluid via the return temperature sensor 54. If the temperature of the return cooling fluid is above a predetermined set point, the computer controller 66 increases the operational speed of the pump-to-pump cooling fluid at a higher rate. If the temperature of the return cooling fluid is below a predetermined set point, the computer controller 66 decreases the operation speed of the pump-to-pump cooling fluid at a lower rate. The computer controller 66 may have a lookup table stored in memory where pump speed is correlated to return cooling fluid temperature. The computer controller 66 may operate the pump 58 at a speed determined from the lookup table and may further increase or decrease the pump speed according to a proportional and/or integral correction factor according to a measured difference in return fluid temperature from a predetermined (desired) return cooling fluid temperature. The temperature indicator 98 is electrically or optically connected to the computer controller. The computer controller 66 may operate the temperature indicator 98 according to the temperature of the return cooling fluid. If the temperature of the return cooling fluid is below a predetermined indicator temperature set point, the computer controller 66 operates the temperature indicator to display a first color of light such as green light. If the temperature of the return cooling fluid is above the indicator temperature set point, the computer controller 66 operates the temperature indicator 98 to display a second color of light, such as red or orange light. In one example, the temperature indicator is an RGB LED or a multicolor LED. In another example, the temperature indicator is a small optical display connected to a light source via a fiber optic cable. The light source may be an LED in or near the pumping unit 10 and the optical display may be a simple lens that disperses the light and makes it visible to the surgeon.

The transplant system 4 may include a disposable transplant set 14. To facilitate easy loading of a transplant set 14, a fluid connector 222 may be provided which facilitates easy connection of an IV bag 214 or other source of sterile saline. An electrical connector 226 may be provided to facilitate easy connection of the temperature sensors 54, 62 and temperature indicator 98. Alternatively, the supply temperature sensor 62 and return temperature sensor 54 may be formed as part of the pumping unit 10 so that the loaded supply tube 50 and return tube 46 are held in contact with the temperature sensors. The pumping unit 10 may include a loading door (such as occupying the front of the pumping unit 10 as drawn) and the disposable set 14 may be loaded into the pumping unit 10 through the loading door. The disposable set may include the cooled transplant pouch 22, temperature indicator 98, supply tube 50, return tube 46, supply temperature sensor 62, return temperature sensor 54, mating portion of connector 226, pump tubing 194, cooling loop 210, reservoir 18 (if used), fill and purge valve(s) 218, and fluid connector 222 formed as a pre-constructed and sterile disposable set 14. The pumping unit 10 may include a cooling section which includes a serpentine channel formed in a cooling block that receives cooling loop 210 and reservoir 18 and which is thermally connected to refrigeration units 70. In FIG. 16, the cooling block would occupy the area adjacent the refrigeration units 70 and would include a channel or recess to receive cooling loop 210 and reservoir 18. The disposable set 14 may be loaded by loading the pump tubing 194 into the peristaltic pump, loading the cooling loop 210 into a channel in the cooling block, properly positioning the fill and purge valve 218 and fluid connector 222, properly positioning the supply tube 50 and return tube 46, connecting the connector 226, and closing the loading door. The transplant system may then be filled with cooling fluid and readied for use.

In another configuration, the system may be similar to that of FIG. 16 and may include a similar disposable set 14 which connects to a source of cooling fluid such as an IV bag with saline 214 and which is similarly loaded into the pumping unit 10. A section of the disposable set tubing may pass through a reservoir holding ice or slush to thereby cool the saline cooling fluid as it circulates through the disposable set. The reservoir may replace the refrigeration units 70 and provide a simple way of cooling the cooling fluid. The cooling fluid may be kept sterile within a closed cooling circuit in the disposable set and non-sterile ice or slush may be used to cool the cooling fluid as the cooling fluid does not contact or mix with the ice or slush.

These transplant systems are advantageous, as they present a simple and reliable system for maintaining the organ 102 at a desired temperature during a transplant surgery. The systems disclosed in FIGS. 14 through 16 may be made at sufficiently low cost to allow the entire system to be disposable. Although there would be some disposal of electronic components, the overall system may still present a reduced cost as compared to sterilizing all or part of a durable transplantation system. The system of FIG. 16 may be disposable or may include a durable pumping unit 10 and disposable transplant set 14.

The transplant system 4 has several advantages over known methods for transplanting an organ. A kidney transplant procedure, without complications, may take significant time. During conventional procedures, the organ is removed from the chilled transport cooler and may warm, leading to greater tissue injury. Even small movements of the organ while holding it during the surgery may result in tissue damage or could tug at newly placed sutures. Additionally, the surgical incision may be small and already crowded by a surgical frame or other equipment used to hold the incision open. Thus, it can be difficult for a surgeon to work comfortably. Transplant procedures for other organs may take even longer than a kidney transplant. Some organs have many attachments, such as veins, arteries, etc., that all must be sutured into correct positions.

The present transplant system 4 eliminates these difficulties in performing the organ transplant. The cooled surgical pouch 22 maintains the desired organ temperature throughout the procedure and thereby reduces tissue damage. The cooled transplant pouch 22 avoids unnecessary organ handling/movement due to supporting and positioning the organ and thereby reduces potential damage to the organ tissue or sutures. The cooled transplant pouch 22 attaches the organ 102 to a surgical support frame at the transplant site, holding the organ steady in a desired position, making it easier to suture and graft the organ. Movement of the organ is reduced and strain on sutures is lessened. The cooled closure straps 146 isolate even the exposed portion of the organ 102 from the body and maintain the chilled organ temperature.

In conventional transplant surgeries, the organ may drip onto the patient or into the incision. While an organ is being held, it is exposed to both dry air and potential contaminants. Presently, an organ must be periodically re-moistened while it is being held, although this tends to result in more dripping into the patient's incision. Additionally, infection after transplantation is a constant concern, and a major cause of organ failure. Although operating rooms are typically equipped with air handling and ventilation systems in order to keep microorganisms to a minimum, such rooms are not perfectly sterile. The patient, healthcare workers, and other objects are all capable of introducing potentially infectious material into the operating room.

The present transplant system 4 minimizes these concerns. With the organ held in the cooled transplant pouch 22, the organ is shielded from the operating room atmosphere. This minimizes drying of the organ and significantly reduces or even eliminates the need to wet the organ. This keeps moistening liquid from dripping into the incision and keeps the surgery site cleaner. Similarly, the enclosed cooling tubes 26, 46, 50 and the enclosed cooling fluid passages 86 keep the cooling fluid completely contained and prevent drips of cooling fluid into the incision. The cooled transplant pouch 22 also shields the organ from any potential contaminants and infectious agents which may be present in the operating room; significantly reducing the risk of infection at the transplant site. The use of a sterilized disposable transplant set 14 also significantly reduces the chance of infection, as it may be reliably sterilized during manufacture and because it minimizes contact between the organ and other objects. In the organ transplant systems, the entire system is sterile and is filled with sterile fluid so any unintended leak which may occur during surgery does not create a contamination concern. Only sterile saline would leak onto the surgical site.

Surgeons must also make note of every piece of equipment entering and leaving an operating room in order to prevent any piece of equipment or supply from being left inside the patient's body. The present transplant system reduces the need for many surgical supplies and, thus reduces the likelihood of foreign matter entering the surgical site. With a reduced or eliminated need to wet the organ and remove dripping liquid from the surgical site, there is reduced usage of surgical supplies and the surgical site is kept cleaner. The disposable transplant set 14 is an integrally formed disposable set and, if used properly, does not generate any loose pieces or debris.

The organ transplant system described herein provides significant advantages over prior art transplantation methods. Optimal conditions for the organ are maintained throughout the surgery. Handling of the organ is minimized while steadiness and positioning of the organ are improved. The transplant system may eliminate the need for sterile slush machines in the operating room and simplifies the equipment and supplies used during the transplant procedure. The transplant system prolongs the time during which optimal organ conditions may be maintained. This improves the surgical outcome and may also allow for additional technologies such as robotic surgeries to be used.

The above description of illustrated examples of the present invention, including what is described in the Abstract, is not intended to be exhaustive or to be limiting to the precise forms disclosed. While specific examples of the invention are described herein for illustrative purposes, various equivalent modifications are possible without departing from the broader scope of the present claims. Indeed, it is appreciated that specific example dimensions, materials, etc., are provided for explanation purposes and that other values may also be employed in other examples in accordance with the teachings of the present invention.

The present application incorporates by references U.S. Provisional Application No. 63/190,684, filed May 19, 2022.

What is claimed is:

1. An organ transplant pouch comprising:
a first layer and a second layer forming a second section and a pathway extending through the center section, and a closure strap extending from the center section, at least a portion of the closure strap having a pathway formed therein.

2. The organ transplant pouch according to claim 1, wherein the pathway in the center section is serpentine and wherein the pathway in the closure strap is serpentine.

3. The organ transplant pouch according to claim 2, wherein the pathway in the closure strap is disposed in communication with the pathway in the center section.

4. The organ transplant pouch according to claim 1, further comprising a grasp tabs extending outwardly from the center section.

5. The organ transplant pouch according to claim 1, wherein the center section has lateral edges and wherein a plurality of grasp tabs extend from the lateral edges.

6. The organ transplant pouch according to claim 1, further comprising a second closure strap extending from the center section, at least a portion of the second closure strap comprises a fluid flow path therethrough.

7. The organ transplant pouch of claim 6 wherein at least one of the closure straps has a cooled portion that is greater than 1 inch long which has a fluid flow path extending therethrough.

8. The organ transplant pouch of claim 7, wherein the cooled portion of at least 2 inches.

9. The organ transplant pouch according to claim 1, wherein the cooled portion of the first closure strap and the cooled portion of the second closure strap slope toward each other in the cooled portion.

10. The organ transplant pouch according to claim 1, wherein the closure strap includes at least one magnetic fastener disposed thereon.

11. An organ transplant system comprising one or more of the following in any combination:
a pumping unit comprising a pump, and a controller for controlling the pump;
tubing including a supply tube and a return tube; and
at least one temperature sensor for detecting temperature of fluid in the return tube.

12. The organ transplant system according to claim 11, further comprising a temperature sensor for detecting temperature of fluid in the supply tube.

13. The organ transplant system according to claim 12, further comprising a transplant pouch attached to the supply tube and the return tube.

14. The organ transplant system according to claim 13, wherein the controller is programmed to compare a detected temperature in the supply tube and a detected temperature in the return to determine a temperature of an organ disposed within the transplant pouch.

15. The organ transplant system according to claim 14, wherein the controller is programmed to adjust a speed at which the pump pumps fluid through the supply tube the temperature of an organ disposed within the transplant pouch falls outside a predetermined temperature range.

16. The organ transplant system of claim 15, further comprising a refrigeration unit disposed in communication with the computer controller such that the controller controls operation of the refrigeration unit to cool cooling fluid.

17. The organ transplant system of claim 16, wherein the controller controls operation of the refrigeration unit in response to one or more of the temperature sensors.

18. The organ transplant system of claim 14, wherein the pouch comprises a fluid inlet, a fluid outlet and a serpentine fluid passage connected to the fluid inlet and connected to the fluid outlet.

19. The organ transplant system of claim 18, wherein the pouch has a closure strap integrally formed therein.

20. The organ transplant system of claim 19, wherein the part of the serpentine fluid passage passes through the closure strap.
